(19) United States
(12) Patent Application Publication
(10) Pub. No.: US 2021/0199226 A1
(43) Pub. Date: Jul. 1, 2021

(54) PROFILE CONNECTION

(71) Applicant: SAINT-GOBAIN PERFORMANCE PLASTICS CORPORATION, Solon, OH (US)

(72) Inventors: Jianfeng ZHANG, Shrewsbury, MA (US); Jian L. Ding, Stow, MA (US); Rachel Z. Pytel, Newton, MA (US)

(21) Appl. No.: 17/127,198

(22) Filed: Dec. 18, 2020

Related U.S. Application Data
(60) Provisional application No. 62/954,193, filed on Dec. 27, 2019.

(51) Int. Cl. F16L 53/34

(52) U.S. Cl. CPC F16L 53/34; A61M 39/18

(57) ABSTRACT

A multi-lumen article includes at least two profiles including at least two lumen, wherein the at least two profiles include a first profile and a second profile, the first profile including a first end and a first lumen, wherein the first lumen provides a fluid flow in a first path; and the second profile including a second end and a second lumen, wherein the second lumen provides a fluid flow in a distinct path different than the first path, wherein at least one profile comprises a polymeric material, wherein the first end and the second end are coincidently bonded without a bonding material at an interface at the first end and the second end.

### CROSS-REFERENCE TO RELATED APPLICATION(S)

This application claims priority under 35 U.S.C. § 119(e) to U.S. Provisional Patent Application No. 62/954,193, entitled “PROFILE CONNECTION”, by Jianfeng ZHANG et al., filed Dec. 27, 2019, which is assigned to the current assignee hereof and incorporated herein by reference in its entirety.

### FIELD OF THE DISCLOSURE

The disclosure, generally, is related to a multi-lumen article and a method providing a multi-lumen article.

### BACKGROUND

Many industries utilize sterile connections for the delivery and removal of fluids. Since sterile connections may be used in a variety of industries, such as the medical industry and pharmaceutical industry, thermoplastic and thermoset elastomers are typically used that are non-toxic, flexible, thermally stable, have low chemical reactivity, and can be produced in a variety of sizes. In many instances, it is desirable to connect at least two different profiles to create a sterile fluid path. Manifolds, in particular, are desirable to provide multi-lumen configurations with minimal fittings. However, it is difficult to effectively provide a weld with a thermoset elastomeric material and in many cases, two different materials, such as two different polymeric materials. For instance, a silicone elastomer is a thermoset material that cannot be melted and thus, cannot be welded with conventional high temperature methods. Further, it is a challenge to maintain any sterility, especially when welding at least two profiles.

Accordingly, an improved multi-lumen article and method of providing a weld between at least two profiles is desired.

### SUMMARY

In an embodiment, a multi-lumen article includes at least two profiles including at least two lumen, wherein the at least two profiles include a first profile and a second profile, the first profile including a first end and a first lumen, wherein the first lumen provides a fluid flow in a first path; and the second profile including a second end and a second lumen, wherein the second lumen provides a fluid flow in a distinct path different than the first path, wherein at least one profile includes a polymeric material, wherein the first end and the second end are coincidently bonded without a bonding material at an interface at the first end and the second end.

In an embodiment, a method of providing a multi-lumen article includes: providing at least a first profile including at least one lumen including a first end and a first lumen; providing at least a second profile comprising a second end and a second lumen, wherein at least the first profile, the second profile, or combination thereof include a polymeric material; providing a surface activation treatment; treating at least the first end, the second end, or combination thereof with the surface activation treatment; and contacting the second end of the second profile directly to the first end of the first profile to coincidently bond the first end to the second end at an interface of the first end and the second end and provide a fluid path, wherein the first lumen has fluid flow in a first path and the second lumen has a fluid flow in a distinct path different than the first path.

### BRIEF DESCRIPTION OF THE DRAWINGS

The present disclosure may be better understood, and its numerous features and advantages made apparent to those skilled in the art by referencing the accompanying drawings.

FIGS. 1A, 1B, and 1C include illustrations of an exemplary multi-lumen article.

FIGS. 2A and 2B include illustrations of an exemplary multi-lumen article.

FIG. 3 includes an illustration of an exemplary multi-lumen article.

The use of the same reference symbols in different drawings indicates similar or identical items.

### DETAILED DESCRIPTION

The following description in combination with the figures is provided to assist in understanding the teachings disclosed herein. The following discussion focuses on specific implementations and embodiments of the teachings. This focus is provided to assist in describing the teachings and should not be interpreted as a limitation on the scope or applicability of the teachings.

As used herein, the terms “comprises”, “comprising”, “includes”, “including”, “has”, “having”, or any other variation thereof, are open-ended terms and should be interpreted to mean “including, but not limited to. . . . ” These terms encompass the more restrictive terms “consisting essentially of” and “consisting of.” In an embodiment, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one or at least one and the singular also includes the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in reference books and other sources within the structural arts and corresponding manufacturing arts. Unless indicated otherwise, all measurements are at about 25° C. For instance, values for viscosity are at 25° C., unless indicated otherwise.

The disclosure generally relates to a multi-lumen article. The multi-lumen article includes at least two profiles including at least two lumen. The at least two profiles include at least a first profile and at least a second profile. In an embodiment, the first profile includes a first end and a first lumen, the first lumen providing a fluid flow in a first path. In an embodiment, the multi-lumen article includes a second profile having a second end and a second lumen, the second lumen providing a fluid flow in a distinct path that is different than the first path. The distinct path of the second lumen that is different than the first path from the first lumen includes any change in fluid flow from the first profile to the second profile such as a change in, for example, a volume, a direction, or any combination thereof. In an embodiment, a fluid flow in a first path having a first direction that includes a linear direction and a fluid flow in a second path may include any change in the linear direction of the first direction. At least one profile includes a polymeric material. A connection is provided by coincidentally bonding the first end of the first profile with the second end of the second profile at an interface of the first end of the first profile and the second end of the second profile. In an embodiment, the coincidental bond is provided via a surface activation treatment. In a particular embodiment, the connection is provided without a bonding material at the interface. Although described as the interface between the first profile and the second profile, “interface” as used herein refers to any coincidently bonded connection and contact point between at least two profiles, which includes an inner surface, an outer surface, an end surface, or combination thereof of the at least two profiles.

In an embodiment, any configuration for each of the at least one profile is envisioned. In an embodiment, the at least one profile has at least one lumen and at least one end. For instance, the first profile may include at least two end, at least three ends, or greater. For instance, the multi-layer article includes a first profile having a first end, a second end, and a third end and a second profile having a second end connected to the first end of the first profile. The multi-lumen article may further include a third profile having a third end, where the third end of the third profile and a second end of the first profile are coincidently bonded without a bonding material at an interface of the third end of the third profile and the second end of the first profile. Further, the multi-lumen article may include a fourth profile having a fourth end, wherein the fourth end of the fourth profile and a third end of the first profile are coincidently bonded without a bonding material at an interface of the fourth end of the fourth profile and the third end of the first profile. Any multi-lumen article with any number of profiles, any number of ends, and any number of lumens is envisioned.

In a particular embodiment, the profile provides a fluid path between at least two lumens for fluid to flow through and between at least two profiles. In an embodiment, the first lumen provides a fluid flow in a first path and the second lumen provides a fluid flow in a second path different than the first path. In an embodiment, a third profile is provided with a third lumen and a third end. In an embodiment, the first end of the first profile, the second end of the second profile, and the third end of the third profile are coincidently bonded at an interface of the first end of the first profile, the second end of the second profile, the third end of the third profile, or combination thereof. In an embodiment, any junction is envisioned at the interface of the profiles. For instance, the at least two coincidently bonded profiles provide a T-junction, a cross-junction, an L-shape, a Y-junction, a star-shape, or combination thereof.

For instance, the profile is any connector, a tube, a port, a hose, a nozzle, a mandrel, a needle, a plug, and the like. Each one of the at least two profiles may be the same or different. In an embodiment, the first profile and the second profile are both tubes. In another embodiment, the first profile is a tube and the second profile is a connector. In an example, each one of the at least two profiles may be a single homogenous polymeric material. In an embodiment, each one of the at least two profiles may be a multi-layered composite material, for example, including more than one distinct polymeric layer.

The surface activation treatment coincidentally and chemically bonds at least two profiles together when they are placed in direct contact. Any surface activation treatment is envisioned and includes any processing input energy to a surface of the at least one profile, such as the first profile, the second profile, or combination thereof. In an embodiment, the processing input energy is with wave irradiation, particle irradiation, or combination thereof. In an embodiment, the wave irradiation includes any wave irradiation envisioned such as radio waves, microwaves, infrared, visible light, ultraviolet, x-rays, gamma radiation, or combination thereof. In a particular embodiment, the wave irradiation includes microwaves, ultraviolet, x-rays, gamma radiation, or combination thereof. In an embodiment, the particle irradiation includes alpha radiation, beta radiation, charged ions, neutron radiation, or combination thereof. In another embodiment, the particle irradiation includes corona treatment, ion treatment, plasma treatment, or combination thereof. In an embodiment, the particle irradiation includes ozone.

The surface activation treatment provides an effective bond, and in a particular embodiment, a seal between the at least two profiles. The efficacy of the seal provides advantageous mechanical and physical properties at the interface of the coincident bond. For instance, the coincident bond withstands a seal integrity pressure test of at least 1 psi, such as at least 5 psi, such as at least 10 psi, such as at least 15 psi, or even at least 20 psi air pressure for about 30 minutes under dry and wet conditions, as described further in the Examples. In an embodiment, the coincident bond maintains a tensile strength of at least about 10%, such as at least about 15%, such as at least about 25%, or even at least about 50%, compared to a tensile strength of a bulk material of each one of the at least two profiles, such as a bulk material of the first profile or a bulk material of the second profile, with the proviso that the comparison is against the bulk material having the lower tensile strength. A measurement of “a bulk material” herein refers to an average measurement obtained over any sampling of the material that is not any portion of the surface that is treated. In a particular embodiment, the coincidental bond has a tensile strength between the at least two profiles, such as the first profile and the second profile, of at least about 10 psi, such as at least about 50 psi, or even at least 300 psi via the tensile test described in the Examples. In an example, the coincident bond maintains an elongation at break of at least about 10%, such as at least about 15%, such as at least about 25%, or even at least about 50%, compared to an elongation at break of a bulk material of each one of the at least two profiles, such as a bulk material of the first profile or a bulk material of the second profile, with the proviso that the comparison is against the bulk material having the lower tensile strength. Furthermore, the coincident bond has a tear strength of at least about 5 ppi, such as at least about 50 ppi, or even at least about 100 ppi, and even at least about 200 ppi via the tear test described in the Examples. In yet another embodiment, the coincident bond has an adhesion force at the interface of at least about 5 ppi, such as at least about 15 ppi, or even as at least about 50 ppi as described via peel test conditions in the Examples.

In an embodiment, the surface treatment provides sterility to the surface it treats, i.e. sterilizes the treated surface. A “treated surface” as used herein refers to any surface that is exposed to surface activation treatment. In an embodiment, “providing sterility” includes maintaining sterility for a pre-sterilized first profile and/or a pre-sterilized second profile. In a particular embodiment, the surface activation treatment provides a sterile connection between each one of the at least two profiles, such as the first profile and the second profile, such as between a treated surface of the first profile and a treated surface of the second profile.

In an embodiment, the first profile includes a first polymeric material. Any polymeric material is envisioned. In an embodiment, the first polymeric material includes a thermoplastic elastomer, a thermoset elastomer, or combination thereof. In a particular embodiment, the first polymeric material is a thermoplastic elastomer and includes a polystyrene, a polyester, a silicone copolymer, silicone thermoplastic vulcanizate, a copolyester, a polyamide, a fluoropolymer, a polyolefin, a polyether-ester copolymer, a thermoplastic urethane, a polyether amide block (PEBA) copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof. In an embodiment, the first polymeric material includes a styrene block copolymer blended with a polyolefin, such as a polypropylene.

In an embodiment, the first polymeric material is a fluoropolymer. An exemplary fluoropolymer includes a copolymer of a poly vinylidene fluoride (PVDF) and a hexafluoropropylene (HFP), a polytetrafluoroethylene (PTFE), a fluorinated ethylene propylene copolymer (FEP), a copolymer of tetrafluoroethylene and perfluoropropyl vinyl ether (PFA), a copolymer of tetrafluoroethylene and perfluoromethyl vinyl ether (MFA), a copolymer of ethylene and tetrafluoroethylene (ETFE), a copolymer of ethylene and chlorotrifluoroethylene (ECTFE), a polychlorotrifluoroethylene (PCTFE), a poly vinylidene fluoride (PVDF), a terpolymer including a tetrafluoroethylene, a hexafluoropropylene, and a vinylidenefluoride (THV), a polyvinyl fluoride (PVF, e.g., Tedlar™), a terpolymer of tetrafluoroethylene, hexafluoropropylene, and ethylene, any blend, any alloy, or combination thereof.

In a particular embodiment, the first polymeric material includes a polyolefin. A typical polyolefin may include a homopolymer, a copolymer, a terpolymer, an alloy, or any combination thereof formed from a monomer, such as ethylene, propylene, butene, pentene, methyl pentene, octene, or any combination thereof. An exemplary polyolefin includes a polyethylene, high density polyethylene (HDPE), medium density polyethylene (MDPE), low density polyethylene (LDPE), ultra or very low density polyethylene (VLDPE), ethylene propylene copolymer, ethylene butene copolymer, polypropylene (PP), polybutene, polybutylene, polypentene, polymethylpentene, polystyrene, ethylene propylene rubber (EPR), ethylene octene copolymer, blend thereof, mixture thereof, and the like. The polyolefin further includes olefin-based random copolymers, olefin-based impact copolymers, olefin-based block copolymers, olefin-based specialty elastomers, olefin-based specialty plastomers, blends thereof, mixture thereof, and the like. In an example, the polyolefin includes polyethylene. In an example, the polyolefin includes polypropylene. In a particular example, the polyolefin is a random propylene copolymer. In an embodiment, the polyolefin is a gamma stabilized polypropylene.

In an additional example, the first polymeric material may include a styrene block copolymer that includes, for example, a multiblock copolymer such as a diblock, triblock, polyblock, or any combination thereof. In a particular embodiment, the styrene block copolymer is a block copolymer having AB units. Typically, the A units are alkenyl arenes such as a styrene, an alpha-methylstyrene, para-methylstyrene, para-butyl styrene, or combination thereof. In a particular embodiment, the A units are styrene. In an embodiment, the B units include alkenes such as butadiene, isoprene, ethylene, butylene, propylene, or combination thereof. In a particular embodiment, the B units are ethylene, isoprene, or combinations thereof. Exemplary styrene block copolymers include triblock styrenic block copolymers (SBC) such as styrene-butadiene-styrene (SBS), styrene-isoprene-styrene (SIS), styrene-ethylene butylene-styrene (SEBS), styrene-ethylene propylene-styrene (SEPS), styrene-ethylene-ethylene-butadiene-styrene (SEEBS), styrene-ethylene-ethylene-propylene-styrene (SEEPS), styrene-isoprene-butadiene-styrene (SIBS), or combination thereof. In an embodiment, the styrene block copolymer is saturated, i.e. does not contain any free olefinic double bonds. In an embodiment, the styrene block copolymer contains at least one free olefinic double bond, i.e. an unsaturated double bond. In a particular embodiment, the styrene block copolymer is a styrene-ethylene based copolymer, a styrene isoprene based copolymer, a blend, or combination thereof.

In an embodiment, the first polymeric material is a thermoset elastomer. Any thermoset elastomer is envisioned. In a particular embodiment, the thermoset elastomer includes a silicone elastomer, a dine elastomer, a butyl rubber, a natural rubber, a polyurethane rubber, an ethylene propylene diene monomer rubber, an isoprene rubber, a nitrile rubber, a styrene butadiene rubber, a blend, or combination thereof. Any rubber for medical/pharmaceutical applications is envisioned. In a particular embodiment, the first polymeric material includes a silicone elastomer.

A typical silicone elastomer includes a silicone matrix component. An exemplary silicone matrix component includes a polyorganosiloxane. Polyorganosiloxanes include a polyalkylsiloxane, a polyarylsiloxane, or combination thereof. Any reasonable polyalkylsiloxane is envisioned. Polyalkylsiloxanes include, for example, silicone polymers formed of a precursor, such as dimethylsiloxane, diethylsiloxane, dipropylsiloxane, methylethylsiloxane, methylpropylsiloxane, or combinations thereof. In a particular embodiment, the polyalkylsiloxane includes a polydialkylsiloxane, such as polydimethylsiloxane (PDMS). In a particular embodiment, the polyalkylsiloxane is a silicone hydride-containing polyalkylsiloxane, such as a silicone hydride-containing polydimethylsiloxane. In a further embodiment, the polyalkylsiloxane is a vinyl-containing polyalkylsiloxane, such as a vinyl-containing polydimethylsiloxane. The vinyl group may be an endblock of the polyalkylsiloxane, on chain of the polyalkylsiloxane, or any combination thereof. In yet another embodiment, the silicone matrix component is a combination of a hydride-containing polyalkylsiloxane and a vinyl-containing polyalkylsiloxane.

In an embodiment, the first polymeric material is a thermoset elastomer and more particularly, a diene elastomer. The diene elastomer may be a copolymer formed from at least one diene monomer. For example, the diene elastomer may be a copolymer of ethylene, propylene and diene monomer (EPDM), a thermoplastic EPDM composite, or combination thereof. An exemplary diene monomer may include a conjugated diene, such as butadiene, isoprene, chloroprene, or the like; a non-conjugated diene including from 5 to about 25 carbon atoms, such as 1,4-pentadiene, 1,4-hexadiene, 1,5-hexadiene, 2,5-dimethyl-1,5-hexadiene, 1,4-octadiene, or the like; a cyclic diene, such as cyclopentadiene, cyclohexadiene, cyclooctadiene, dicyclopentadiene, or the like; a vinyl cyclic ene, such as 1-vinyl-1-cyclopentene, 1-vinyl-1-cyclohexene, or the like; an alkylbicyclononadiene, such as 3-methylbicyclo-(4,2,1)-nona-3,7-diene, or the like; an indene, such as methyl tetrahydroindene, or the like; an alkenyl norbornene, such as 5-ethylidene-2-norbornene, 5-butylidene-2-norbornene, 2-methallyl-5-norbornene, 2-isopropenyl-5-norbornene, 5-(1,5-hexadienyl)-2-norbornene, 5-(3,7-octadienyl)-2-norbornene, or the like; a tricyclodiene, such as 3-methyltricyclo (5,2,1,0²,6)-deca-3,8-diene or the like; or any combination thereof.

Depending on the composition of the first polymeric material, the first polymeric material may be formed with any reasonable component such as any precursor with the addition of any reasonable additive. An additional additive includes, but is not limited to, a catalyst, a filler, a plasticizer, a lubricant, an antioxidant, a colorant, an optically transparent conductive additive, an adhesion promoter, heat stabilizer, acid scavenger, UV stabilizer, processing aid, or combination thereof. In a particular embodiment, the precursor, the additional additive such as the catalyst, the filler, plasticizer, lubricant, antioxidant, colorant, an optically transparent conductive additive, an adhesion promoter, heat stabilizer, acid scavenger, UV stabilizer, processing aid, or combination thereof are dependent upon the first polymeric material chosen and final properties desired for the first profile.

Any reasonable catalyst that can initiate crosslinking of the polymeric material is envisioned. Exemplary catalysts include a catalyst that may be heat cured, IR radiation cured, e-beam cured, or combination thereof. The catalyst is dependent upon the polymeric material chosen. The catalyst may or may not be used in combination with a crosslinker promoter, such as triallyl cyanurate (TAC), triallyl isocyanurate (TAIC), or combination thereof. In an embodiment, the additive includes any reasonable adhesion promoter. Any reasonable adhesion promoter that promotes adhesion of adjacent layers is envisioned and is dependent upon the adjacent layers. Exemplary lubricants include silicone oil, waxes, slip aids, antiblock agents, the like, or any combination thereof. Exemplary lubricants further include silicone grafted polyolefin, polyethylene or polypropylene waxes, Oleic acid amide, erucamide, stearate, fatty acid esters, the like, or any combination thereof. Exemplary antioxidants include phenolic, hindered amine antioxidants. Exemplary fillers include calcium carbonate, talc, radio-opaque fillers such as barium sulfate, bismuth oxychloride, any combinations thereof, and the like. In an embodiment, the filler includes a functionalized filler. Exemplary functionalized fillers include, for example, a base filler that has a functional moiety that forms a chemical bond with the second polymeric material. Any reasonable base filler is envisioned such as a silica filler, fumed silica filler, quartz, glass filler, aluminum (AO(OH)), alumino-silicate, inorganic oxides, resinous filler, carbon black, graphite, graphene, carbon nanotube (CNT), fullerene or combination thereof. In a particular embodiment, the functionalized filler includes a silica filler. Any functional moiety is envisioned that has an adhesive affinity to the second polymeric material. The functionalized moiety is, for example, a silane attached to the base filler, wherein the silane includes an acryl functional group, an epoxy functional group, a chloro functional group, or combination thereof. In an embodiment, any reasonable silane is envisioned and includes, for example, an alkoxysilane such as a trimethoxysilane, a triethoxysilane, or combination thereof. In an embodiment, the functionalized filler is a silicone-hydride attached to the base filler. In a particular embodiment, the silicone-hydride is trimethylsiloxy-terminated. When present as the functional moiety, any reasonable amount of functionalized filler is envisioned to provide an increased adhesive bond between the first polymeric material and the second polymeric material. In an embodiment, the functionalized filler forms a cohesive bond between the first polymeric material and the second polymeric, i.e. cohesive failure occurs wherein the structural integrity of the first profile and/or the second profile fails before the bond between the two materials fails. In an exemplary embodiment, the functionalized filler is mixed with the polymeric material to form a homogenous mixture of the functionalized filler contained with a matrix of the polymeric material. In an embodiment, the functionalized filler may or may not form a reactive and covalent bond with the polymeric material. In a more particular embodiment, the functionalized filler does not form a reactive and covalent bond with the polymeric material. Exemplary plasticizers include any known plasticizers such as a citrate, a phthalate, a trimellitate, 1,2-cyclohexane dicarboxylic acid diisonoyl ester (DINCH), an adipate, a polymeric plasticizer, a castor oil, a caster oil derivative, mineral oils, soybean oil, such as epoxidized soybean oil, the like, or any combination thereof.

Typically, the additional additive may be present at an amount of not greater than about 70% by weight of the total weight of the polymeric material, such as not greater than about 60% by weight of the total weight of the polymeric material, such as not greater than about 50% by weight of the total weight of the polymeric material, such as not greater than about 40% by weight of the total weight of the polymeric material, or even not greater than about 30% by weight of the total weight of the polymeric material. In an alternative embodiment, the polymeric material may be substantially free of an additional additive such as a catalyst, lubricant, a filler, a plasticizer, an antioxidant, a colorant, an adhesion promoter, heat stabilizer, acid scavenger, UV stabilizer, processing aid, or combination thereof. “Substantially free” as used herein refers to less than about 1.0% by weight, or even less than about 0.1% by weight of the total weight of the polymeric material.

Further included is at least a second profile. The second profile includes a second polymeric material, a metal, or combination thereof. In an embodiment, the second polymeric material includes a thermoplastic elastomer, a thermoset elastomer, or combination thereof as described for the first polymeric material. In an embodiment, each one of the at least two profiles, such as the first polymeric material and the second polymeric material, are the same polymeric material. In an embodiment, the multi-lumen article consists of one polymeric material. In another embodiment, each one of the at least two profiles, such as the first polymeric material and the second polymeric material, are different polymeric materials. For instance, the connection of the multi-lumen article may be between any combination of the first polymeric material and the second polymeric material being: a silicone elastomer, a styrene block copolymer, a polyvinyl chloride, a fluoropolymer, a polyolefin, a polycarbonate, a diene copolymer, a blend, or combination thereof. In an embodiment, the first polymeric material and/or the second polymeric material include a silicone elastomer, a styrene block copolymer blended with a polyolefin, a polyvinyl chloride, a polytetrafluoroethylene (PTFE), a fluorinated ethylene propylene copolymer (FEP), a copolymer of tetrafluoroethylene and perfluoropropyl vinyl ether (PFA), a polyethylene, a polycarbonate, a polyolefin, a diene copolymer, a blend, or combination thereof. In an embodiment, the first polymeric material and/or the second polymeric material are a silicone elastomer, a styrene block copolymer blended with a polyolefin, or combination thereof. In an embodiment, the second profile includes a metal. Any metal is envisioned. In a particular embodiment, the second profile is a stainless steel.

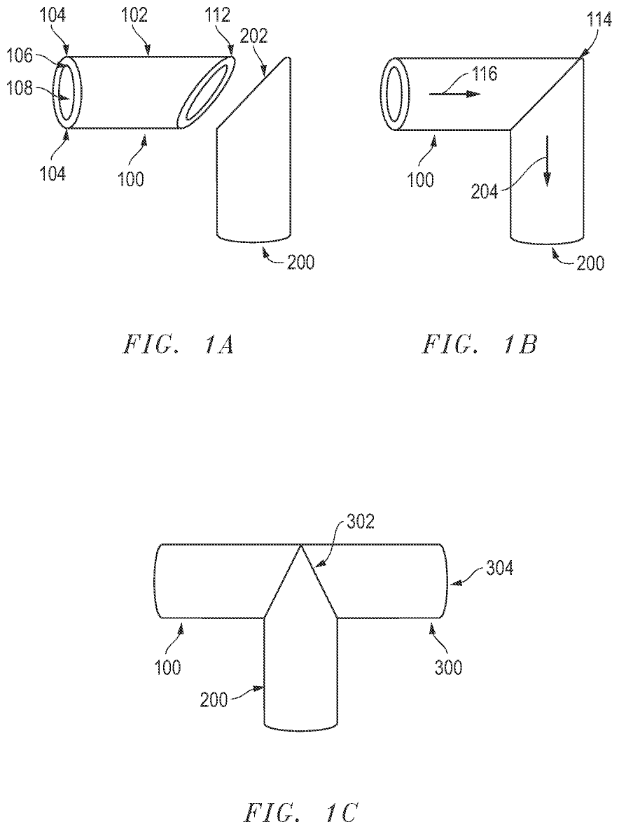

FIG. 1A is a view of a first profile 100 and a second profile 200 according to an embodiment. Typically, the first profile 100 and the second profile 200 is any commercially available profile. In a particular embodiment, the first profile 100 is in the form of a tube including a body 102 having an outside diameter 104 and an inner diameter 106. The inner diameter 106 can form a hollow bore 108 of the body 102. The hollow bore 108 defines a lumen of the tube for fluid flowthrough. In addition, the body 102 is illustrated as a single layer, the single layer including the first polymeric material. The body 102 can include a wall thickness that is measured by the difference between the outside diameter 104 and the inner diameter 106.

In a particular embodiment, the outside diameter 104 of the body 102 is about 0.025 inches to about 5.0 inches, such as about 0.15 inches to about 2.0 inches. It will be appreciated that the outside diameter 104 can be within a range between any of the minimum and maximum values noted above. In an embodiment, the inner diameter 106 of the body 102 is about 0.005 inches to about 4.0 inches, such as about 0.06 inches to about 1.0 inches. It will be appreciated that the inner diameter 106 can be within a range between any of the minimum and maximum values noted above. The wall thickness is about 0.02 inches to about 4.0 inches, such as about 0.05 inches to about 1.0 inch, or even about 0.1 inches to about 0.5 inches. It will be appreciated that the wall thickness 110 can be within a range between any of the minimum and maximum values noted above. Further, the body 102 has a first end 112.

Although the cross-section of the inner bore 108 perpendicular to an axial direction of the body 102 in the illustrative embodiment shown in FIG. 1A has a circular shape, the cross-section of the inner bore 108 perpendicular to the axial direction of the body 102 can have any cross-section shape envisioned.

In a particular embodiment, the second profile 200 is in the form of a tube similarly described for first profile 100 and can include a body having an outside diameter and an inner diameter. The inner diameter can form a hollow bore of the body. The hollow bore defines a central lumen of the tube for fluid flowthrough. In addition, the body may be a single layer, the single layer including the second polymeric material. The body can include a wall thickness that is measured by the difference between the outside diameter and the inner diameter.

In a particular embodiment, the outside diameter of the body is about 0.025 inches to about 5.0 inches, such as about 0.15 inches to about 2.0 inches. It will be appreciated that the outside diameter can be within a range between any of the minimum and maximum values noted above. In an embodiment, the inner diameter of the body is about 0.005 inches to about 4.0 inches, such as about 0.06 inches to about 1.0 inches. It will be appreciated that the inner diameter can be within a range between any of the minimum and maximum values noted above. The wall thickness is about 0.02 inches to about 4.0 inches, such as about 0.05 inches to about 1.0 inch, or even about 0.1 inches to about 0.5 inches. It will be appreciated that the wall thickness can be within a range between any of the minimum and maximum values noted above. Further, the second profile 200 has a second end 202.

Although the cross-section of the inner bore perpendicular to an axial direction of the body of the second profile 200 in the illustrative embodiment shown in FIG. 1A may have a circular shape, the cross-section of the inner bore perpendicular to the axial direction of the body can have any cross-section shape envisioned.

Although illustrated as a single layer tube for both the first profile 100 and the second profile 200, any number of layers is envisioned. For instance, the first profile and the second profile include one layer, two layers, three layers, or even a greater number of layers. Further, although illustrated as both tubes with substantially the same inner diameter, outer diameter, and wall thickness, each one of the at least two profiles, such as the first profile 100 and the second profile 200, can have the same or different configurations. Irrespective of the number of layers present, the outside diameter and inner diameter of the first profile 100 and the second profile 200 can have any values as defined for the single layer tubes 100, 200 defined in FIG. 1A. The number of layers is dependent upon the final properties desired for the multi-lumen article. Further, although illustrated as a single lumen, i.e. a hollow bore for both the first profile 100 and the second profile 200, any number of lumen is envisioned. For instance, the first profile and/or the second profile include a plurality of lumen.

In an embodiment, the first profile 100, the second profile, 200, or combination thereof may further include other layers. Other layers include, for example, a polymeric layer, a reinforcing layer, an adhesive layer, a barrier layer, a chemically resistant layer, a metal layer, any combination thereof, and the like. Any additional layer is envisioned and is dependent upon the material chosen. In an embodiment, any number of polymeric layers is envisioned.

In an embodiment, a method of providing a multi-lumen article is provided. The method includes providing at least a first profile 100 including at least one lumen including a first end 112 and a first lumen. The method further includes providing the second profile 200 having the second end 202 and the second lumen. In an embodiment, at least the first profile 100, the second profile 200, or combination thereof is cut. Any method of cutting is envisioned. In a particular embodiment and as seen in FIG. 1B, the first end 112 and the second end 202 are coincidently bonded together via a surface activation treatment, the interface of the coincidental bond having an exterior seam 114. For instance, the surface activation treatment is provided to treat a surface of the first end 112 and a surface of the second end 202 and the first end 112 and the second end 202 are placed in direct contact to coincidentally bond the first end 112 to the second end 202. Typically, a compression force of less than 100 Newtons (N) is applied to the abutting first end 112 and second end 202. The fluid flow through the first profile 100 is in a first path 116 and the fluid flow through the second profile 200 is in a distinct path 204 that is different than the first path 116. As illustrated, the distinct path 204 is in a direction different than the first path 116. As illustrated, the coincident bond provides an L-shape. In an embodiment and as illustrated, the first and second profiles are both tubes, coincidentally bonded to provide two different fluid path directions. However, in an alternative embodiment, at least on profile includes a connector, such as an elbow joint, and a tube coincidently bonded therewith to provide two different fluid path directions.

For instance and as seen in FIG. 1C, the multi-lumen article includes a third profile having a third end 302 and a third lumen 304. The third end 302 of the third profile 300 may be bonded to the first end 112 of the first profile 100, the second end 202 of the second profile 200, or combination thereof to coincidently bond the first end 112, the second end 202, and the third end 302 to form, for example, a T-junction. As illustrated, both the third end 302 of the third profile 300 and the first end 112 of the first profile 100 are coincidently bonded to the second end 202 of the second profile 200 to form a T-junction. Although not illustrated, the multi-lumen article includes a Y-junction.

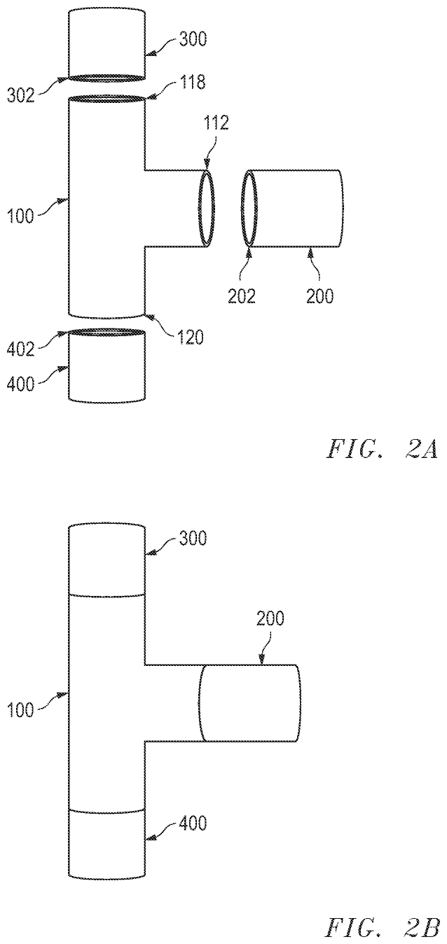

According to an embodiment, FIG. 2A is a view of a first profile 100 and a second profile 200, and a third profile 300. Typically, the first profile 100, the second profile 200, and the third profile 300 are any commercially available profile. In a particular embodiment, the first profile 100 is in the form of a multi-lumen article including at least three ends, a first end 112 of the first profile 100, a second end 118 of the first profile 100, and a third end 120 the first profile 100. The second profile 200 is in the form of a tube as described with FIGS. 1A-1C and has a second end 202. The third profile 300 is in the form of a tube as described with FIGS. 1A-1C and has a third end 302. The first end 112 of the first profile 100 may be coincidently bonded to the second end 202 of the second profile 200. The second end 118 of the first profile 100 may be coincidently bonded to the third end 302 of the third profile 300. A fourth profile 400 is in the form of a tube and has a fourth end 402. The third end 120 of the first profile 100 may be coincidently bonded to the fourth end 402 of the fourth profile 400. Although illustrated as a first profile 100 with at least three ends 112, 118, and 120, any number of ends is envisioned. As seen in FIG. 2B, the first profile 100 is coincidently bonded to the the second profile 200, the third profile 300, and the fourth profile 400.

In a particular embodiment, a portion of the first end of the first profile has a surface that is in direct contact and coincidently bonded with a portion of a surface of the second end of the second profile. In an embodiment, the portion of the surface of at least one profile coincidently bonded to another profile includes the inner surface, the outer surface, the end surface, or combination thereof. In a particular embodiment, the first profile, the second profile, or combination thereof has a desirable surface roughness (Ra) to provide a desirable seal. For instance, the treated surface of the first profile 100 and second profile 200, such as a cross-section across the wall thickness 110 of the first profile 100, the cross-section across the wall thickness 210 of the second profile, or combination thereof has a Ra of less than about 20 μm, such as less than about 5 sm, such as less than about 1 sm, or even less than about 0.5 μm, as measured by a MarSurf M 300C Mobile Roughness Measuring Instrument. In an example, the surface activation treatment minimally changes a surface roughness of a treated surface. In an embodiment, the surface roughness of a treated surface of each one of the at least two profiles changes by less than about 5%, such as less than about 2%, or even less than about 1%, compared to an untreated surface of each one of the at least two profiles, such as the first profile and the second profile.

The interface has further advantageous physical and chemical properties. In an embodiment, the interface has a mechanical strength of at least 2%, such as at least 10%, or even at least 35% of a bulk material of each one of the at least two profiles, such as the first profile and the second profile, with testing conditions as described by the tensile test in the Examples. For instance, the interface has a failure mode of cohesive failure. Although not being bound by theory, a surface activation treatment at least excites an atom at a molecular level to provide the coincident bond. For instance, the treated surface has an oxygen atomic concentration of greater than about 2%, such as greater than about 5%, such as greater than about 10%, or even greater than about 15%, compared to a bulk material of each one of the at least two profiles, such as a bulk material of the first profile and a bulk material of the second profile via XPS. For instance, the treated surface has an nitrogen atomic concentration of greater than about 2%, such as greater than about 5%, such as greater than about 10%, or even greater than about 15%, compared to a bulk material of each one of the at least two profiles, such as the first profile and the second profile via XPS. In a particular embodiment, the interface has a higher valence of an element, compared to a bulk material of each one of the at least two profiles, such as a bulk material of the first profile and a bulk material of the second profile. For instance, the treated surface has a surface tension of greater than about 20, such as greater than about 22, or even greater than about 25, as described by the surface energy test in the Examples. In particular, a surface tension is increased at a treated interface for connection and/or treated surface for at least about 1 mM/m, at least about 3 mM/m, or even at least than about 10 mM/m, as described by the surface energy test in the Examples.

In an embodiment, the coincidental bond 114 is a circumferential seal wherein the bonded ends, 112 and 202, are abutted. In a particular embodiment, the bonded ends 112 and 202 maintain fluid flow through the hollow bore 108 of profile 100 and through profile 200. Although discussed as bonded ends, any surface of each one of the two profiles can be bonded, such as the inner surface, the outer surface, the end surface, or combination thereof of the at least two profiles. For instance, an inner surface of at least a first profile may be bonded to an outer surface of at least a second profile. In an embodiment, an outer surface of at least a first profile may be bonded to an inner surface of at least a second profile. Any combination of surfaces to be bonded may be envisioned.

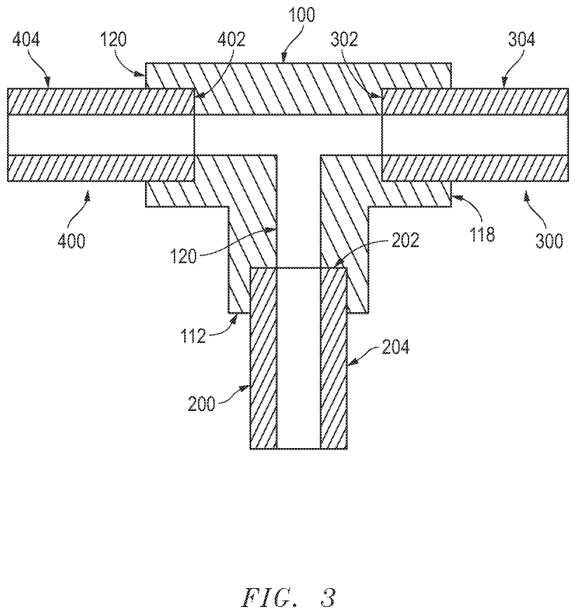

As seen in FIG. 3, an exemplary internal view of a first profile 100, a second profile 200, a third profile 300, and a fourth profile 400 is illustrated. Typically, the first profile 100, the second profile 200, the third profile 300, and the fourth profile 400 are any commercially available profile. In a particular embodiment, the first profile 100 is in the form of a multi-lumen article including at least three ends, a first end 112 of the first profile 100, a second end 118 of the first profile 100, and a third end 120 the first profile 100. The second profile 200 is in the form of a tube as described with FIGS. 1A-1C and has a second end 202. The third profile 300 is in the form of a tube as described with FIGS. 1A-1C and has a third end 302. As illustrated, an outside surface 204 and end 202 of the second profile 200 is directly contact with and coincidently bonded to an inside surface 120 of first profile 100. Further, an outside surface 304 and end 302 of the third profile 300 is directly contact with and coincidently bonded to an inside surface 120 of first profile 100. Further, an outside surface 404 and end 402 of the fourth profile 400 is directly contact with and coincidently bonded to an inside surface 120 of first profile 100. Although illustrated as a first profile 100 with at least three ends 112, 118, and 120, any number of ends is envisioned.

The coincident bond provides an advantageous seal between at least the first profile 100 and the second profile 200. In an embodiment, the bond between at least two profiles provides multi-lumen configurations. In a particular embodiment, the bond at the interface has a desirable integrity with the tensile test. For instance, the integrity of the interface via the tensile test is equal to or better than a standard, commercially available fitted joint. Typically, the interface is substantially free of a bonding material. Any bonding material includes any external adhesive material envisioned such as any added material that provides adhesive properties. Furthermore, the interface is substantially free of any reversible chemistry. “Reversible chemistry” as used herein refers to a chemical reaction that forms a new chemical compound that is different than an original chemical compound. Furthermore and in an embodiment, the surface activation does not increase a temperature of a treated surface to exceed the melting point of the bulk material.

In a particular embodiment, a sterile connection is provided between each one of the at least two profiles, such as the first profile 100 and the second profile 200. In an embodiment, each one of the at least two profiles, such as at least the first profile 100, the second profile 200, or combination thereof, are sterile prior to the coincident bond. In an embodiment, the surface activation treatment provides a sterile connection between each one of the at least two profiles, such as the first profile 100 and the second profile 200, or at least maintains sterility of a pre-sterilized each one of the at least two profiles, such as a pre-sterilized first profile 100 and/or a pre-sterilized second profile 200. In an embodiment, the surface activation treatment provides a sterile connection between the treated surface of the first profile 100 and the treated surface of the second profile 200 or at least maintains sterility of a treated surface of a pre-sterilized first profile 100 and/or a treated surface of a pre-sterilized second profile 200. In an embodiment, the surface activation treatment sterilizes each one of the at least two profiles, such as the treated surface of the first profile 100, the treated surface of the second profile 200, or combination thereof. Although not illustrated, the surface activation treatment may be used to provide a visible bubble at the interface. The visible bubble may be advantageous as a visual indicator that a seal has been achieved or when the seal is no longer present.

As described, the surface activation treatment includes, in an embodiment, corona treatment, plasma treatment, ion treatment, or combination thereof. For instance, the corona treatment ionizes the atmosphere to activate a surface of each one of the at least two profiles, such as the first profile and the second profile. In an embodiment, the surface activation treatment includes plasma treatment such as, for example, an inert gas plasma, an oxygen-containing plasma, a nitrogen-containing plasma, a fluorine-containing plasma, or combination thereof. In an embodiment, the surface activation treatment includes plasma treatment which ionizes a gas such as helium, neon, oxygen, argon, nitrogen, compressed air, ammonia, or combination thereof. In an embodiment, the surface activation treatment includes plasma treatment which ionizes a gas such as oxygen, argon, nitrogen, compressed air, ammonia, or combination thereof. Any conditions of the surface activation treatment are envisioned that provides a bond as well as sterile conditions for the at least two profiles, such as the first profile 100 and the second profile 200. For instance, the plasma treatment is provided for less than 2 minutes, such as less than 1 minute, such as less than 45 seconds, such as less than 30 seconds, or even less than 10 seconds. In a particular embodiment, an extraction profile of each one of the at least two profiles, such as the first profile and the second profile, before and after surface activation treatment is substantially identical, indicating that the chemical composition of each one of the at least two profiles, such as the first profile and the second profile, has not changed before and after surface activation treatment. Furthermore, a change in particulates in each one of the at least two profiles, such as the first profile and the second profile, before and after surface activation treatment is +/−5%, such as +/−15%, or even +/−50%. In an embodiment, the profiles may be surface treated multiple times. For instance, the method can include disconnecting the coincident bond at the interface, providing an additional surface activation treatment, and contacting the first end directly to the second end to coincidently bond the first end to the second end at the interface.

Since the surface treatment provides sterility to each one of the at least two profiles, such as the first profile 100 and the second profile 200, a further sterilization process is not required. Further, the surface activation treatment provides an effective seal where the coincidental bond is substantially free of an adhesive, a primer, a chemical treatment, or combination thereof. Any energy, dependent on power and time, is envisioned that activates the surface of the first profile and the second profile. For examples, a power output is about 480 Watts for about 5 seconds.

In an embodiment, a reinforcement (not illustrated) can be used to reinforce the exterior seam 114. In an embodiment, the reinforcement is a fastening device that surrounds at least a portion of the exterior seam of the coincidental bond. In a particular embodiment, the fastening device that surrounds the entire exterior seam of the coincidental bond. Any fastening device is envisioned such as, for example, a clamp, a polymer tape, an overmolded polymer, a glue, or combination thereof. In a particular embodiment, the fastening device is a polymer tape such as a silicone tape. The silicone tape may be self-adhesive. In another embodiment, a surface between the polymer tape is surface treated to enhance the adhesion of the polymer tape to an exterior surface adjacent to the coincidental bond. For instance, the surface of the polymer tape is treated.

In another embodiment, the outer surface of the exterior seam is treated. In a particular embodiment, the surface between the polymer tape is surface treated with the surface activation treatment described for the bonding and sterilizing of each one of the at least two profiles, such as the first profile and the second profile. Any sequence of surface treating the polymer tape concurrently or subsequently with surface treatment with the surface activation treatment for bonding/welding is envisioned. In an embodiment, the fluid path is substantially free of an external physical connector, a thermal weld connection, or combination thereof.

In exemplary embodiments, each one of the at least two profiles with the coincidental bond can be used in a variety of applications where a bonded connection is desired. In a particular embodiment, a sterile connection is achieved. Advantageously and in a particular embodiment, the surface activation treatment provides a method of bonding and sterilizing a multitude of polymeric materials not yet before bonded/welded while maintaining a sterilized connection. In particular, the sterile nature of the coincidental bond is useful for any application where sterility is desired. For instance, the coincidental bond of any profiles has potential for FDA, ADCF, USP Class VI, NSF, European Pharmacopoeia compliant, United States Pharmacopoeia (USP) compliant, USP physiochemical compliant, ISO 10993 Standard for evaluating biocompatibility of a medical device, and other regulatory approvals. In a particular embodiment, the profile is non-cytotoxic, non-hemolytic, non-pyrogenic, animal-derived component-free, non-mutagenic, non-bacteriostatic, non-fungistatic, or any combination thereof.

In an embodiment, the method of providing a multi-lumen article may be used in applications such as industrial, medical applications, health care, biopharmaceutical, drinking water, food & beverage applications, dairy applications, laboratory applications, FDA applications, and the like. In an exemplary embodiment, the method of providing a multi-lumen article may be used in applications such as a fluid transfer tube in food and beverage processing equipment, a fluid transfer tube in medical and health care, biopharmaceutical manufacturing equipment, and peristaltic pump tube for medical, laboratory, and biopharmaceutical applications.

In a particular embodiment, a fluid source, such as a container, reactor, reservoir, tank, or bag, is coupled to each one of the at least two profiles, such as the first profile and/or the second profile. For instance, the first profile and/or the second profile may engage a pump, fitting, valve, dispenser, or another container, reactor, reservoir, tank, or bag. In an example, the first profile and/or the second profile may be coupled to a water container and may have a dispenser fitting. In another example, the first profile and/or the second profile may be coupled to a fluid bag and coupled to a valve. In a further example, the profile may be coupled to a container, be engaged in a pump, and be coupled to a second container.

Many different aspects and embodiments are possible. Some of those aspects and embodiments are described herein. After reading this specification, skilled artisans will appreciate that those aspects and embodiments are only illustrative and do not limit the scope of the present invention. Embodiments may be in accordance with any one or more of the items as listed below.

Embodiment 1. A multi-lumen article including at least two profiles including at least two lumen, wherein the at least two profiles include a first profile and a second profile, the first profile including a first end and a first lumen, wherein the first lumen provides a fluid flow in a first path; and the second profile including a second end and a second lumen, wherein the second lumen provides a fluid flow in a distinct path different than the first path, wherein at least one profile includes a polymeric material, wherein the first end and the second end are coincidently bonded without a bonding material at an interface at the first end and the second end.

Embodiment 2. The multi-lumen article of embodiment 1, wherein the first profile includes at least three ends; and further including a third profile having a third end; wherein the third end of the third profile and a second end of the first profile are coincidently bonded without a bonding material at an interface of the third end and the second end.

Embodiment 3. The multi-lumen article of embodiment 2, further including a fourth profile having a fourth end, wherein the fourth end of the fourth profile and a third end of the first profile are coincidently bonded without a bonding material at an interface of the fourth end and the third end.

Embodiment 4. The multi-lumen article of embodiment 1, further including a third profile having a third end, wherein the first end of the first profile, the second end of the second profile, the third end of the third profile, or combination thereof are coincidently bonded.

Embodiment 5. A method of providing a multi-lumen article including: providing at least a first profile including at least one lumen including a first end and a first lumen; providing at least a second profile including a second end and a second lumen, wherein at least the first profile, the second profile, or combination thereof include a polymeric material; providing a surface activation treatment; treating at least the first end, the second end, or combination thereof with the surface activation treatment; and contacting the second end of the second profile directly to the first end of the first profile to coincidently bond the first end to the second end at an interface of the first end and the second end and provide a fluid path, wherein the first lumen has fluid flow in a first path and the second lumen has a fluid flow in a distinct path different than the first path.

Embodiment 6. The multi-lumen article or method of providing the multi-lumen article of any of the preceding embodiments, wherein the first profile coincidently bonded to the second profile provides a T-junction, a cross-junction, a L-shape, a Y-junction, a star-shape, or combination thereof.

Embodiment 7. The multi-lumen article or method of providing the multi-lumen article of any of the preceding embodiments, wherein the first profile, the second profile, or combination thereof include a polymeric material, a metal, or combination thereof.

Embodiment 8. The multi-lumen article or method of providing the multi-lumen article of embodiment 7, wherein first polymer material and the second polymeric material are the same polymeric material.

Embodiment 9. The multi-lumen article or method of providing the multi-lumen article of embodiment 7, wherein the first polymeric material and the second polymeric material are different polymeric materials.

Embodiment 10. The multi-lumen article or method of providing the multi-lumen article of any of the preceding embodiments, wherein the polymeric material includes a thermoplastic elastomer, a thermoset elastomer, or combination thereof.

Embodiment 11. The multi-lumen article or method of providing the multi-lumen article of embodiment 10, wherein the thermoplastic elastomer includes a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

Embodiment 12. The multi-lumen article or method of providing the multi-lumen article of embodiment 11, wherein the thermoset elastomer includes a silicone elastomer, a diene elastomer, a butyl rubber, a natural rubber, a polyurethane rubber, an ethylene propylene diene monomer rubber, an isoprene rubber, a nitrile rubber, a styrene butadiene rubber, a blend, or combination thereof.

Embodiment 13. The multi-lumen article or method of providing the multi-lumen article of any of the preceding embodiments, wherein the at least one profile includes a silicone elastomer tube.

Embodiment 14. The multi-lumen article or method of providing the multi-lumen article of any of the preceding embodiments, wherein the at least one profile includes a tubing, a receiver, a connector, a hose, a needle, a nozzle, or combination thereof.

Embodiment 15. The multi-lumen article or method of providing the multi-lumen article of any of the preceding embodiments, wherein the coincidental bond is a circumferential seal.

Embodiment 16. The multi-lumen article or method of providing the multi-lumen article of any of the preceding embodiments, wherein the coincident bond withstands a seal integrity pressure test of at least 1 psi, such as at least 5 psi, such as at least 10 psi, such as at least 15 psi, or even at least 20 psi air pressure for about 30 minutes under dry and wet conditions.

Embodiment 17. The multi-lumen article of any of the preceding embodiments, wherein the coincident bond is provided via surface activation treatment.

Embodiment 18. The multi-lumen article or method of providing the multi-lumen article of embodiment 17, wherein the surface activation treatment includes processing input energy to a surface of the first profile, the second profile, or combination thereof with wave irradiation, particle irradiation, or combination thereof.

Embodiment 19. The multi-lumen article or method of providing the multi-lumen article of embodiment 18, wherein the wave irradiation includes microwaves, ultraviolet, x-rays, gamma radiation, or combination thereof.

Embodiment 20. The multi-lumen article or method of providing the multi-lumen article of embodiment 18, wherein the particle irradiation includes alpha radiation, beta radiation, charged ions, neutron radiation, or combination thereof.

Embodiment 21. The multi-lumen article or method of providing the multi-lumen article of embodiment 18, wherein the particle irradiation includes corona treatment, ion treatment, plasma treatment, or combination thereof.

Embodiment 22. The multi-lumen article or method of providing the multi-lumen article of embodiment 21, wherein the plasma treatment is provided for less than 2 minutes, such as less than 1 minute, such as less than 45 seconds, such as less than 30 seconds, or even less than 10 seconds.

Embodiment 23. The multi-lumen article or method of providing the multi-lumen article of any of the preceding embodiments, wherein the at least one profile has a wall thickness of about 0.02 inches to about 4.0 inches, such as about 0.05 inches to about 1.0 inch, or even about 0.1 inches to about 0.375 inches.

Embodiment 24. The multi-lumen article or method of providing the multi-lumen article of any of the preceding embodiments, wherein the second profile has a wall thickness of about 0.02 inches to about 4.0 inches, such as about 0.05 inches to about 1.0 inch, or even about 0.1 inches to about 0.375 inches.

Embodiment 25. The multi-lumen article or method of providing the multi-lumen article of any of embodiments 23 and 24, wherein the first profile and the second profile have the same wall thicknesses.

Embodiment 26. The multi-lumen article or method of providing the multi-lumen article of any of embodiments 23 and 24, wherein the first profile and the second profile have different wall thicknesses.

Embodiment 27. The multi-lumen article or method of providing the multi-lumen article of any of the preceding embodiments, wherein the first profile has an inner diameter that is the same as an inner diameter of the second profile.

Embodiment 28. The multi-lumen article or method of providing the multi-lumen article of any of embodiments 1-26, wherein the first profile has an inner diameter that is different than an inner diameter of the second profile.

Embodiment 29. The multi-lumen article or method of providing the multi-lumen article of any of the preceding embodiments, wherein the first profile has an outer diameter that is the same as an outer diameter of the second profile.

Embodiment 30. The multi-lumen article or method of providing the multi-lumen article of any of embodiments 1-28, wherein the first profile has an outer diameter that is different than an outer diameter of the second profile.

Embodiment 31. The multi-lumen article or method of providing the multi-lumen article of any of the preceding embodiments, wherein the coincidental bond has a tensile strength between the first profile and the second profile of at least about 10 psi, such as at least about 50 psi, or even at least 300 psi.

Embodiment 32. The multi-lumen article or method of providing the multi-lumen article of any of the preceding embodiments, wherein the profile is used for biopharm applications, FDA applications, medical applications, laboratory applications, or combination thereof.

Embodiment 33. The multi-lumen article or method of providing the multi-lumen article of any of the preceding embodiments, wherein a fastening device surrounds at least a portion of an exterior seam of the coincidental bond.

Embodiment 34. The multi-lumen article or method of providing the multi-lumen article of embodiment 33, wherein the fastening device includes a clamp, a polymer tape, an overmolded polymer, a glue, or combination thereof.

Embodiment 35. The multi-lumen article or method of providing the multi-lumen article of any of the preceding embodiments, wherein the surface activation treatment provides a sterile connection of a treated surface of the at least one profile.

Embodiment 36. The method of providing the multi-lumen article of embodiment 5, further including providing a third profile comprising a third end and a third lumen; and contacting the third end of the third profile directly to the first end of the first profile and the second end of the second profile to coincidently bond the first end, the second end, and the third end.

Embodiment 37. The multi-lumen article or method of providing the multi-lumen article of any of the preceding embodiments, wherein the coincident bond maintains a tensile strength of at least about 10%, such as at least about 15%, such as at least about 50%, or even at least about 100%, compared to a tensile strength of a bulk material of the first profile or a bulk material of the second profile, with the proviso that the comparison is against the bulk material having the lower tensile strength.

Embodiment 38. The multi-lumen article or method of providing the multi-lumen article of any of the preceding embodiments, wherein the coincident bond has an adhesion force at the interface of at least 5 psi, such as at least 50 psi, at least of 100 psi, or even at least of 200 psi.

Embodiment 39. The multi-lumen article or method of providing the multi-lumen article of any of the preceding embodiments, wherein a failure mode at the interface of the coincident bond is adhesive failure.

Embodiment 40. The multi-lumen article or method of providing the multi-lumen article of any of the preceding embodiments, wherein a surface roughness of a treated surface of the first profile and a surface roughness of a treated surface of the second profile changes by less than about 5%, such as less than about 2%, or even less than about 1%, compared to an untreated surface of the first profile and an untreated surface of the second profile.

Embodiment 41. The multi-lumen article or method of providing the multi-lumen article of any of the preceding embodiments, wherein a failure mode at the interface of the coincident bond is cohesive failure.

Embodiment 42. The multi-lumen article or method of providing the multi-lumen article of any of the preceding embodiments, wherein a surface of the first profile at the interface contains chemical components from a surface of the second profile at the interface.

Embodiment 43. The multi-lumen article or method of providing the multi-lumen article of any of the preceding embodiments, wherein a surface of the second profile at the interface contains chemical components from a surface of the first profile at the interface.

Embodiment 44. The multi-lumen article or method of providing the multi-lumen article of any of the preceding embodiments, wherein the treated surface has an oxygen atomic concentration of greater than about 2%, such as greater than about 5%, such as greater than about 10, or even greater than about 15%, compared to a bulk material of the first profile and a bulk material of the second profile via XPS.

Embodiment 45. The multi-lumen article or method of providing the multi-lumen article of any of the preceding embodiments, wherein the treated surface has a higher valence of an element compared to a bulk material of the first profile and a bulk material of the second profile.

Embodiment 46. The multi-lumen article or method of providing the multi-lumen article of any of the preceding embodiments, having a surface tension at a treated surface of greater than about 20, such as greater than about 22, or even greater than about 25.

Embodiment 47. The multi-lumen article or method of providing the multi-lumen article of any of the preceding embodiments, wherein the interface is substantially free of a bonding material.

Embodiment 48. The multi-lumen article or method of providing the multi-lumen article of any of the preceding embodiments, further including a visible bubble at the interface.

Embodiment 49. The multi-lumen article or method of providing the multi-lumen article of any of the preceding embodiments, wherein a fluid path is substantially free of an external physical connector, a thermal weld connection, or combination thereof.

Embodiment 50. The multi-lumen article or method of providing the multi-lumen article of embodiment 49, wherein the multi-lumen article consists of one polymeric material.

The concepts described herein will be further described in the following examples, which do not limit the scope of the disclosure described in the claims. The following examples are provided to better disclose and teach processes and compositions of the present invention. They are for illustrative purposes only, and it must be acknowledged that minor variations and changes can be made without materially affecting the spirit and scope of the invention as recited in the claims that follow.

### Examples

General Procedure for Welding and Burst Test:

Welding: placed the two tubes under plasma, exposed the cross-sections to plasma for a certain time; then immediately after the treatment, aligned the tubes and “weld” them by applying gentle compression force (less than 100N but making sure the ends are in fill contact).

Post treatment of tubing: the welded tubing were stored in ambient temperature and pressure for certain period before connecting to compression air for burst pressure test.

Burst Test Pressure Procedures

The pressure was provided via connecting to a compression air line with a regulator to control the pressure during the test. One end of the tested “welded” tubing was connected to the regulator using braid reinforcing silicone tubing with proper fitting. The other end of the “welded” tubing was connected to a pressure gauge. The whole tubing was immersed in water during the test. The fail of the tubing (burst at the joint or burst of tubing) could be easily indicated by the air bubble in the water tank. When the test began, the pressure was increased by controlling the regulator with the rate about 2 psi/s. The highest pressure during the test was recorded. The whole process was also recorded by video and confirmed all the readings were correct after test.

Standard Operating Procedure of the test:

1. Connected the welded tubing to the test apparatus.

2. Laid the pressure testing apparatus on a flat surface.

3. Filled the water tank with enough water to submerge test specimens.

4. Connected pressure testing apparatus to a clean, dry compressed air supply.

5. Determined the correct multi-barb fitting sizes for the tubing to be tested. Slightly oversized barbed fittings were acceptable as long as they did not cause the tubing to leak at the barbed fitting.

6. Installed both end of the specimen on to the barbed fitting and secured with at least 1 zip tie.

7. Slowly pressurized the apparatus (˜2 psi/s) until air bubbles were observed in the water tank.

8. Cleaned and dried the apparatus to repeat the test as necessary, in general, at least 3 samples were tested for one condition.

Two different burst types were recorded:

1) Where the tube inflated with the increase of the pressure from the compression air, and burst at the joint at the highest pressure (named here as Type A burst).

2) Where the tubing inflated with the increase of the pressure from the compression air, and then the tubing materials yielded, the tubing bulged; however the pressure dropped, then tubing broke at the joint, the burst pressure was lower than the highest pressure during the test (named here as Type B burst).

Test 1:

Materials: 50 shore A durometer silicone tubing “welding” with same 50 shore A durometer silicone tubing (½ inch ID, % inch OD). Results can be seen in Table 1.

TABLE 1

| | Plasma | Storage time | Highest | |
|---|---|---|---|---|
| | treatment time | at ambient | pressure during | Burst |
| | (seconds) | condition | test (psi) | type |
| 1-1* | 5 | 1 minute | 25 | A |
| 1-2* | 5 | 3 minute | 75 | A |
| 1-3* | 5 | 5 minute | 31.5 | A |
| 1-4* | 5 | 15 minute | 32 | A |
| 1-5 | 5 | 2 hour | 34.6 ± 0.6 | A |
| 1-6^{#} | 5 | 2 hour | 38 | B |
| 1-7* | 5 | 2 days | 34 | A |
| 1-8 | 10 | 2 hour | 32.8 ± 0.9 | A |
| 1-9 | 15 | 2 hour | 31.3 ± 1 | A |

*Only one sample was tested at this condition. For the rest of the tests, at least 2 samples were tested.
^{#}The coincident bond was reinforced with a layer of silicone tape (wrapping with plasma induced adhesion).

Test 2:

The following materials were tested: A 65 shore A durometer silicone tubing “welding” with the same 65 shore A durometer silicone tubing (½ inch ID, % inch OD). Results can be seen in Table 2.

TABLE 2

| | Plasma | Storage time | Highest | |
|---|---|---|---|---|
| | treatment time | at ambient | pressure during | Burst |
| | (seconds) | condition | test (psi) | type |
| 7.4* | 5 | 2 h | 50.5 | A |
| 2-2*^{#} | 5 | 2 h | 49 | B |

*Only one sample was tested at this condition. For the rest of the tests, at least 3 samples were tested.
^{#}The joint was reinforced with a layer of silicone tape (wrapping with plasma induced adhesion).

Test 3:

The following materials were tested: a silicone tubing “welding” with C-Flex tubing (½ inch ID, % inch OD). Shore A durometer of the silicone tubing was seen in Table 3. Results can be seen in Table 3.

TABLE 3

| | | Plasma | Plasma | | Highest | |
|---|---|---|---|---|---|---|
| | | treatment | treatment | Storage | pressure | |
| | | time | time | time | during | |
| | Silicone | at silicone | at C-Flex | at ambient | test | Burst |
| | hardness | (seconds) | (seconds) | condition | (psi) | type |
| 3-1* | 50 | 5 | 5 | 2 hour | 23 | A |
| 3-2* | 50 | 5 | 15 | 2 hour | 21.5 | A |
| 3-3*^{#} | 65 | 5 | 50 | 2 hour | 45 | A |

*Only one sample was tested at this condition. For the rest of the tests, at least 3 samples were tested.
^{#}The joint was reinforced with a layer of silicone tape (wrapping with plasma induced adhesion).

Tensile Test—Non-ASTM Standard

Plasma treatment conditions are in Table 4 along with max strain, tensile strength, and measured by the following procedure.

Preparation of the sample: for the as-is tubing/control, the tubing was cut with a length 4-5 inch; for welded tubing, the tubing after welding was-4-5 inch with the welding line locating at the middle.

Placed the tubing in the Instron tensile test machine with both ends into grips. The gas between the grips was set at 2 inch, making sure the grips were securely holding the tubing sample.

Pulled the sample with the tensile machine at a rate of 20 in/min until tubing break, the grips were pulled to 20 inches apart, or until the maxima tensile range of the machine was reached.

Removed the sample from the tensile machine and inspect for visual failure. Calculated the strength based on the ring area of tubing cross-section.

All durometer was shore A. Control samples were tubes un-cut tubes.

TABLE 4

| | | | Plasma | Plasma | | |
|---|---|---|---|---|---|---|
| | | | treatment | treatment | | Max |
| | | | time at A | time at B | Max | tensile |
| | Tubing | Tubing | tubing | tubing | strain | strength |
| | A | B | (sec.) | (sec.) | (%) | (psi) |
| 1 | 50 duro | C-Flex | 5 | 20 | 43 ± 2 | 134 ± 5 |
| | silicone | #1 | | | | |
| 2* | 65 duro | C-Flex | 5 | 20 | 22 | 106 |
| | silicone | #1 | | | | |
| 3* | 50 duro | C-Flex | 5 | 35 | 45 | 133 |
| | silicone | #1 | | | | |
| 4* | 65 duro | C-Flex | 5 | 35 | 28 | 127 |
| | silicone | #1 | | | | |
| 5* | 65 duro | C-Flex | 5 | 50 | 48 | 103 |
| | silicone | #1 | | | | |
| 6 | 65 duro | 65 duro | 5 | 5 | 25 ± 2 | 112 ± 8 |
| | silicone | silicone | | | | |
| 7^{#} | 65 duro | 65 duro | 5 | 5 | 73 ± 50 | 215 ± 84 |
| | silicone | silicone | | | | |
| 8 | 50 duro | 50 duro | 5 | 5 | 47 ± 6 | 116 ± 11 |
| | silicone | silicone | | | | |
| 9^{#} | 50 duro | 50 duro | 5 | 5 | 283 ± 32 | 345 ± 38 |
| | silicone | silicone | | | | |
| 10^{&} | 50 duro silicone control | | | | >581 ± 60 | >581 ± 50 |
| 11^{&} | 65 duro silicone control | | | | >811 ± 8 | >893 ± 4 |
| 12 | C-Flex #1 control | | | | >1332 | >824 |
| 13 | C-Flex #1 thermal welding | | | | 98 | 266 |
| 14 | C-Flex | C-Flex | 60 | 60 | 35 | 137 |
| | #1 | #1 | | | | |
| 15 | C-Flex #2 control | | | | | |
| 16 | C-Flex #2 thermal welding | | | | 520 | 2510 |
| 17 | C-Flex | C-Flex | 60 | 60 | 35 | 40 |
| | #2 | #2 | | | | |

*Only one sample was tested at this condition. For the rest of test condition, at least 3 samples were tested.
^{#}The joint was reinforced with a layer of silicone tape (wrapping with plasma induced adhesion).
^{&}The control sample did not break in the middle of the tubing, test terminated either when sample slipped from clamps or broke from the defect due to clamping.

The same tensile test conditions were used to determine the tear strength and mechanical strength. Results for tear strength can be seen in Table 5.

TABLE 5

| | 65 duro Silicone | 65 duro Silicone |
|---|---|---|
| | Control Uncut | plasma welded to |
| | tube | 65 duro Silicone |
| Tear strength (ppi) | 897 | 281 |

Burst Pressure Test—Impact of Processing Parameter:

Materials: 50 duro silicone tubing “welding” with same 50 duro silicone tubing (½ inch ID, ¾ inch OD)

Burst pressure tests were performed with at least 2 hours after welding. Results can be seen in Table 6.

TABLE 6

| | | Argon flow (SLPM) | | |
|---|---|---|---|---|
| Burst pressure (psi) | | 2.8-3.0 | 6.0-6.3 | 15.0-15.3 |
| Treatment | 5 | 29.6 ± 1.7 | 32.3 ± 0.4 | 18.3 ± 5.5 |
| time (s) | 10 | 34.6 ± 0.6 | 32.8 ± 0.9 | 31.3 ± 1 |
| | 15 | 33.3 ± 1.7 | 33.6 ± 1 | 29.3 ± 0.9 |

Surface Tension was Tested Via the Following Conditions:

Plasma welding surface energy of C-Flex and silicone tubing after exposure to plasma for welding procedure conditions and results were as follows.

Description of the tested materials is seen in Table 7.

TABLE 7

| Reference | Description |
|---|---|
| C-flex control | Control C-flex sample (no plasma) |
| C-flex plasma weld | C-flex, adjacent to plasma weld mark |
| Silicone control | Control silicone samples (no plasma) |
| Silicone plasma weld | Silicone, adjacent to plasma weld mark |

ASTM D7334-08, “Standard Practice for Surface Wettability of Coatings, Substrates and Pigments by Advancing Contact Angle Measurement” was followed. This practice deals with the measuring of contact angles to characterize the wettability of surfaces. Two different solvents were used: water and diiodomethane (MI).

The instrument used was a Kruss Mobile Surface Analyzer, which uses an automatic liquid dispenser to place drops of solvent (volume=˜1 μL) on a sample. Drops of water and MI were placed in parallel and allowed to settle on the surface. The values of the two contact angles were determined using drop shape analysis. 5+ drops of each solvent were tested on each sample surface.

For analysis, the Owens-Wendt method was used, which utilizes both the dispersive and polar components of each solvent to determine the surface energy components of the samples. The equation for the method follows:

$$\frac{{σ}_{L} ( cos θ + 1 )}{2 {\sqrt{σ}}_{L}^{D}} = \frac{{\sqrt{σ}}_{S}^{P} {\sqrt{σ}}_{L}^{P}}{{\sqrt{σ}}_{L}^{D}} + {\sqrt{σ}}_{S}^{D}$$

Where: cos θ: Cosine of the contact angle of the liquid drop on the sample;

σ_{L}: Surface tension of the liquid;

σ_{L}^{D}: Dispersive component of the surface tension of the liquid;

σ_{L}^{P}: Polar component of the surface tension of the liquid;

σ_{S}^{D}: Dispersive component of the surface energy of the sample;

σ_{S}^{P}: Polar component of the surface energy of the sample.

The equation fits a linear equation y=mx+b. By fitting a linear regression using the mean contact angle of each drop and liquid surface tension components, the surface energy components of the sample was determined.

Contact angle measurements and surface energy calculations are shown in Table 8 and 9 below. Measurements are taken on the treated surface.

TABLE 8
Contact angle measurements

| | Measurement | | | |
|---|---|---|---|---|
| Sample | Mean Water | | Mean MI | |
| ID | CA (°) | STD | CA (°) | STD |
| C-flex control | 100.4 | 2.6 | 56.8 | 8.8 |
| C-flex plasma weld | 72.4 | 13.2 | 44.3 | 6.1 |
| Silicone control | 101.6 | 5.7 | 98.5 | 3.6 |
| Silicone plasma weld | 93.9 | 6.5 | 73.0 | 3.8 |

TABLE 9
Surface energy calculations

| | Surface Energy | | | | | |
|---|---|---|---|---|---|---|
| | Surface Free | | Dis- | | | |
| Sample | Energy | | persive | | Polar | |
| ID | [mN/m] | STD | [mN/m] | STD | [mN/m] | STD |
| C-flex control | 30.8 | 5.5 | 30.4 | 5.0 | 0.3 | 0.4 |
| C-flex plasma | 44.4 | 9.3 | 37.4 | 3.3 | 7.0 | 6.0 |
| weld | | | | | | |
| Silicone control | 13.6 | 3.5 | 9.2 | 1.3 | 4.4 | 2.2 |
| Silicone plasma | 24.3 | 4.2 | 21.2 | 2.1 | 3.0 | 2.1 |
| weld | | | | | | |

Peel Test Conditions were as Follows:

The adhesion strength was measured by the following procedure. Preparation of the sample: Two silicone slabs with ˜ 1/16 inch thick were stacked and welded by plasma. The welded silicone slabs was cut into ¼ inch wide pieces. The welded slabs were then placed in the instron with each slab gripped, and peeled with T shape/180 degree peel. The peel force was 9.9±3.9 ppi.

Extraction profile was determined as follows: The control silicone tubing and welded silicone tubing were extracted using 50% water and 50% of ethanol for 24 hours at 70° C. Then Gas Chromatograph/Mass Spectrometry was used to analyze the extraction profile. Notably, plasma welding did not substantially change the extraction profile of a material, such as silicone tubing. In an example, when comparing a silicone control and a plasma welded silicone, plasma welding did not increase the extraction of siloxanes.

Note that not all of the activities described above in the general description or the examples are required, that a portion of a specific activity may not be required, and that one or more further activities may be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. A multi-lumen article comprising at least two profiles comprising at least two lumen, wherein the at least two profiles comprise a first profile and a second profile, the first profile comprising a first end and a first lumen, wherein the first lumen provides a fluid flow in a first path; and
the second profile comprising a second end and a second lumen, wherein the second lumen provides a fluid flow in a distinct path different than the first path, wherein at least one profile comprises a polymeric material, wherein the first end and the second end are coincidently bonded without a bonding material at an interface at the first end and the second end.

2. The multi-lumen article in accordance with claim 1, wherein the first profile comprises at least three ends; and
further comprising a third profile having a third end; wherein the third end of the third profile and a second end of the first profile are coincidently bonded without a bonding material at an interface of the third end and the second end.

3. The multi-lumen article in accordance with claim 2, further comprising a fourth profile having a fourth end, wherein the fourth end of the fourth profile and a third end of the first profile are coincidently bonded without a bonding material at an interface of the fourth end and the third end.

4. The multi-lumen article in accordance with claim 1, further comprising a third profile having a third end, wherein the first end of the first profile, the second end of the second profile, the third end of the third profile, or combination thereof are coincidently bonded.

5. The multi-lumen article in accordance with claim 1, wherein the first profile, the second profile, or combination thereof comprise a polymeric material, a metal, or combination thereof.

6. The multi-lumen article in accordance with claim 1, wherein the polymeric material comprises a thermoplastic elastomer, a thermoset elastomer, or combination thereof.

7. The multi-lumen article in accordance with claim 6, wherein the thermoplastic elastomer comprises a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

8. The multi-lumen article in accordance with claim 1, wherein the at least one profile comprises a silicone elastomer tube.

9. The multi-lumen article in accordance with claim 1, wherein the coincident bond withstands a seal integrity pressure test of at least 1 psi, such as at least 5 psi, such as at least 10 psi, such as at least 15 psi, or even at least 20 psi air pressure for about 30 minutes under dry and wet conditions.

10. The multi-lumen article in accordance with claim 1, wherein the coincident bond is provided via surface activation treatment.

11. The multi-lumen article in accordance with claim 10, wherein the surface activation treatment comprises processing input energy to a surface of the first profile, the second profile, or combination thereof with wave irradiation, particle irradiation, or combination thereof.

12. The multi-lumen article in accordance with claim 10, wherein the surface activation treatment provides a sterile connection of a treated surface of the at least one profile.

13. A method of providing a multi-lumen article comprising:
providing at least a first profile comprising at least one lumen comprising a first end and a first lumen;
providing at least a second profile comprising a second end and a second lumen, wherein at least the first profile, the second profile, or combination thereof comprise a polymeric material;
providing a surface activation treatment;
treating at least the first end, the second end, or combination thereof with the surface activation treatment; and
contacting the second end of the second profile directly to the first end of the first profile to coincidently bond the first end to the second end at an interface of the first end and the second end and provide a fluid path, wherein the first lumen has fluid flow in a first path and the second lumen has a fluid flow in a distinct path different than the first path.

14. The method of providing the multi-lumen article in accordance with claim 13, wherein the first profile, the second profile, or combination thereof comprise a polymeric material, a metal, or combination thereof.

15. The method of providing the multi-lumen article in accordance with claim 13, wherein the polymeric material comprises a thermoplastic elastomer, a thermoset elastomer, or combination thereof.

16. The method of providing the multi-lumen article in accordance with claim 15, wherein the thermoplastic elastomer comprises a polystyrene, a polyester, a silicone copolymer, a silicone thermoplastic vulcanizate, a copolyester, a polyamide, a fluoropolymer, a polyolefin, a polyether-ester copolymer, a thermoplastic urethane, a polyether amide block copolymer, a polyamide copolymer, a styrene block copolymer, a polycarbonate, a thermoplastic vulcanizate, an ionomer, a polyoxymethylene (POM), an acrylonitrile butadiene styrene (ABS), an acetal, an acrylic, a polyvinyl chloride (PVC), a blend, or combination thereof.

17. The method of providing the multi-lumen article in accordance with claim 13, wherein the at least one profile comprises a silicone elastomer tube.

18. The method of providing the multi-lumen article in accordance with claim 13, wherein the coincident bond withstands a seal integrity pressure test of at least 1 psi, such as at least 5 psi, such as at least 10 psi, such as at least 15 psi, or even at least 20 psi air pressure for about 30 minutes under dry and wet conditions.

19. The method of providing the multi-lumen article in accordance with claim 13, wherein the surface activation treatment comprises processing input energy to a surface of the first profile, the second profile, or combination thereof with wave irradiation, particle irradiation, or combination thereof.

20. The method of providing the multi-lumen article in accordance with claim 13, wherein the surface activation treatment provides a sterile connection of a treated surface of the at least one profile.
